(19) United States
(12) Patent Application Publication
(10) Pub. No.: US 2014/0194850 A1
(43) Pub. Date: Jul. 10, 2014

(54) ACCESSORY FOR A SYRINGE

(71) Applicant: Cyrille Vinchon, Chilly (FR)

(72) Inventor: Cyrille Vinchon, Chilly (FR)

(73) Assignee: ANTEIS SA, Meyrin (CH)

(21) Appl. No.: 14/127,290

(22) PCT Filed: Jul. 2, 2012
(86) PCT No.: PCT/EP2012/062866

§ 371 Date: Feb. 11, 2014

(30) Foreign Application Priority Data
Jul. 4, 2011 (EP) 11172585.9

(51) Int. Cl. A61M 5/34

(52) U.S. Cl. CPC A61M 5/344
USPC 604/506; 604/240

(57) ABSTRACT

An accessory for a syringe is disclosed, including a body with a proximal end having at least one zone for application of an injection force in order to apply a force for discharging a product from the syringe, and a distal end having a bearing element for bearing against a needle mounted on the mouth of the syringe with a force that includes a radial component perpendicular to the longitudinal axis of the syringe and directed towards the centre of the syringe. An assembly including such an accessory and a syringe is also disclosed.

### PRIORITY STATEMENT

This application is the national phase under 35 U.S.C. §371 of PCT International Application No. PCT/EP2012/062866 which has an International filing date of Jul. 2, 2012, which designated the United States of America and which claims priority to European patent application number EP 11172585.9 filed Jul. 4, 2011, the entire contents of each of which are hereby incorporated herein by reference.

### FIELD

At least one embodiment of the present invention generally relates to an accessory for a syringe. At least one embodiment of the present invention relates in particular for an accessory for holding a needle or a cannula on a syringe in order to avoid any risk of the needle or the cannula becoming inadvertently detached when a product is injected.

### BACKGROUND

Needles or cannulas are constituted of a hub, generally of plastic material or of metal, and of a tube, generally metallic, that can be sharpened or polished in the case of needles or cannulas. The hub generally has a conical geometry of the female “luer” connector type, inspired for example from standard ISO594 “Conical fittings with a 6% (Luer) taper for syringes, needles and certain other medical equipment”, the contents of which is incorporated herein by reference. The syringe is equipped with a nozzle having a conical geometry of the male “luer” connector type, inspired from standard ISO594 “Conical fittings with a 6% (Luer) taper for syringes, needles and certain other medical equipment”. The assembly and tightening of the 2 cones thus formed make it possible to create adhesion and to hold the needle or cannula on the syringe's nozzle.

An injection made by means of a syringe generally has the effect of generating a longitudinal stress on the needle or the cannula during injection. In some cases, notably during the injection of a strongly viscous product and/or by means of a long and/or small-caliber needle or cannula, this stress risks resulting in the needle and/or its connecting fitting becoming separated relative to the syringe's body during the injection. This is called a detachment of the needle.

A connection by screwing, called “luer lock”, partly remedies this problem by ensuring a good connection between the needle and the syringe, but even so an unscrewing of the needle under said stress cannot be excluded. Such an unscrewing, even partial, can cause lesions to the neighboring tissues due to the displacement or sudden and uncontrolled expulsion of the needle. This problem arises for example in the field of cosmetic or reconstructive surgery, during which viscous products are used, as well as in various therapeutic fields (ophthalmology, rheumatology, urology, devices for controlled-release of active substances . . . ). Furthermore, in the case of a luer lock that is an integral part on a syringe body, the longitudinal stress on the needle can result in the needle and the luer lock becoming completely detached if the stress against the needle during injection exceeds a certain threshold.

An accessory for a syringe is described in international application WO2004/007006 by Becton Dickinson France. This accessory aims to hold the needle onto the syringe during injection by tightening the needle against the syringe. In order to achieve this tightening, this accessory proposes a first holding means for bearing against the needle and a second holding means for bearing against the proximal end of the syringe body. This accessory has the disadvantage of being dependent on its own rigidity: if it deforms easily, it is easy to assemble onto the syringe and the needle but does not allow the needle to be held efficiently in the case of longitudinal stress and great deformation. In contrast, if it is poorly deformable, the syringe and its needle are difficult to mount into this accessory and it adapts poorly to the different models of available needles or cannulas. Finally, this patent application also describes rotation-blocking elements in order to counter the detaching through unscrewing. Such an indexation no longer enables the user to orient the bevel, the elbow or the exit hole of the needle or cannula in the desired direction in the tissues.

### SUMMARY

One aim of the present invention is thus to propose an accessory for a syringe that makes it possible to prevent any detaching of the needle during injection, whatever the linear stress exerted on the needle.

Another aim of the present invention is to propose an accessory for a syringe that can be installed in an easy manner on the syringe, without risk of the needle becoming contaminated.

Another aim of the present invention is to propose an accessory for a syringe that can be used together with a mechanically assisted and/or automatic injection system enabling injection pain during use of fine needles or cannulas to be reduced whilst maintaining usual injection speeds.

Yet another aim of the invention is to propose an accessory for a syringe that prevents any detaching of the needle during injection and that is easy to install whilst not having the disadvantages or limitations of the prior art accessories.

These aims and other advantages are achieved by an accessory for a syringe and by an assembly comprising such an accessory and a syringe, for manual use or by means of an assisted injection system, comprising the characteristics of the corresponding independent claims.

These aims and other advantages are achieved in particular by an accessory for a syringe comprising a body with a proximal end having at least one zone for application of a holding force in reaction to an injection force on the piston of the syringe for applying a pressure to discharge a product outside of said syringe and a distal end comprising a bearing element for bearing against a needle mounted on the nozzle of the syringe with a force comprising a radial component perpendicular to the longitudinal axis of said syringe and directed towards the center of said syringe.

These aims and other advantages are achieved also by an assembly comprising such an accessory, a syringe and a needle or a cannula.

These aims and other advantages are also achieved by an assembly comprising such an accessory, a syringe and a needle or a cannula, and a mechanical and/or automatic assisted injection system.

### BRIEF DESCRIPTION OF THE DRAWINGS

The present invention will be better understood by reading the following description, illustrated by the figures in which:

FIG. 1 is a perspective view of an accessory for a syringe according to one embodiment of the invention;

FIG. 2 is a cross section of a syringe and of the accessory of FIG. 1 ready for the injection;

FIG. 3 is a detail of FIG. 2;

FIG. 4 is a cross section of the syringe and of the accessory of the preceding figures in an initial position before they are prepared for injection;

FIG. 5 is a perspective view of the syringe and of the accessory in the position illustrated in FIG. 4;

FIG. 6 is a perspective view of a syringe and of an accessory according to another embodiment of the invention;

FIG. 7 illustrates diagrammatically an assisted injection device onto which the syringe and the accessory of FIG. 6 are fastened.

### DETAILED DESCRIPTION OF THE EXAMPLE EMBODIMENTS

Exerting a radial bearing force on the needle enables the adhesion force between the needle, especially its hub, and the needle's nozzle to be reinforced, whilst considerably reducing the risk of reaching the limits of the needle's mechanical resistance, in particular of its hub, and thus of damaging it during the injection.

According to a variant embodiment, the accessory's proximal end comprises a housing surrounding at least part of said syringe in its entire periphery.

According to one embodiment, the bearing element comprises an essentially cylindrical element and makes it possible for example to bear on the needle's hub on a significant portion of its circumference, for example on a quarter of this circumference. According to a variant embodiment, this cylindrical element can deform upon contact of the needle's hub and thus conform to its tapered and/or cylindrical geometry.

According to one embodiment, the bearing element comprises a tapered element that matches the needle's hub and makes it possible for example to bear on the needle, in particular on the needle's hub, on approximately half of its circumference, for example on two thirds of this circumference.

According to one embodiment, the distal end comprises an opening on its circumference enabling the needle to be inserted in a direction perpendicular to the longitudinal axis of the syringe, thus allowing the syringe to rotate relative to the accessory from an initial position wherein the nozzle of the syringe is accessible for the needled to be mounted and an injection position wherein the end of the syringe is in contact with the distal end of the accessory, whose bearing element bears on the needle to ensure it is held on the syringe during the injection.

According to one embodiment, the distal end comprises a housing for receiving at least part of a luer lock placed on the syringe without the luer lock being in contact with the distal end when the bearing element bears against the needle, thus preventing a force from being transmitted from the accessory to the luer lock. In a similar manner, the housing of the accessory's proximal end makes it possible to avoid any significant transmission of the force necessary to the injection, exerted on the accessory for example by a user, for example on the collar of the syringe that usually serves to transmit the injection force. The force exerted by the user or by the assisted injection device on the accessory to perform the injection is thus transmitted by the accessory's distal end onto the needle, for example onto its hub, to ensure that it is held on the syringe.

The accessory of the present invention thus makes it possible to completely avoid any detaching of the needle, since the force transmitted by the accessory to the needle is generally greater than the force exerted on the needle by the product contained in the syringe and discharged through it. This is achieved as soon as the inner diameter of the syringe body is greater than the application diameter of the gel's pressure on the needle at the level of the syringe's nozzle, which is the case for the great majority of syringes.

Since the accessory for a syringe according to the invention bears directly on the needle's hub and furthermore comprises a zone for application of a holding force as a reaction to the injection force, comprising for example bearing surfaces for the fingers of a user during injection and/or fastening means of the accessory onto an assisted injection device, the force applied to the accessory for a syringe by the user or by the assisted injection device during injection is essentially transmitted to the needle's hub, holding the latter firmly against the syringe with a force at least equal to, generally greater than the stress exerted against the needle by the product discharged out of the syringe.

According to a variant embodiment, the accessory bears onto the needle's hub in a direction parallel to the axis of the syringe opposite to the stress exerted on the needle by the product discharged out of the syringe, and also on the conical and/or cylindrical walls of the needle, in particular of its hub, in a radial direction towards the center of the needle, thus tightening the latter firmly against the extremity of the syringe.

According to a variant embodiment, the accessory for a syringe is particularly adapted for being used with a fixed or integral luer lock and the part that bears against the needle to hold it onto the syringe comprises an essentially conical part that is inserted into the luer lock when the accessory is in place for the injection. The accessory is then locked into position, thus preventing it from moving away from the syringe during injection.

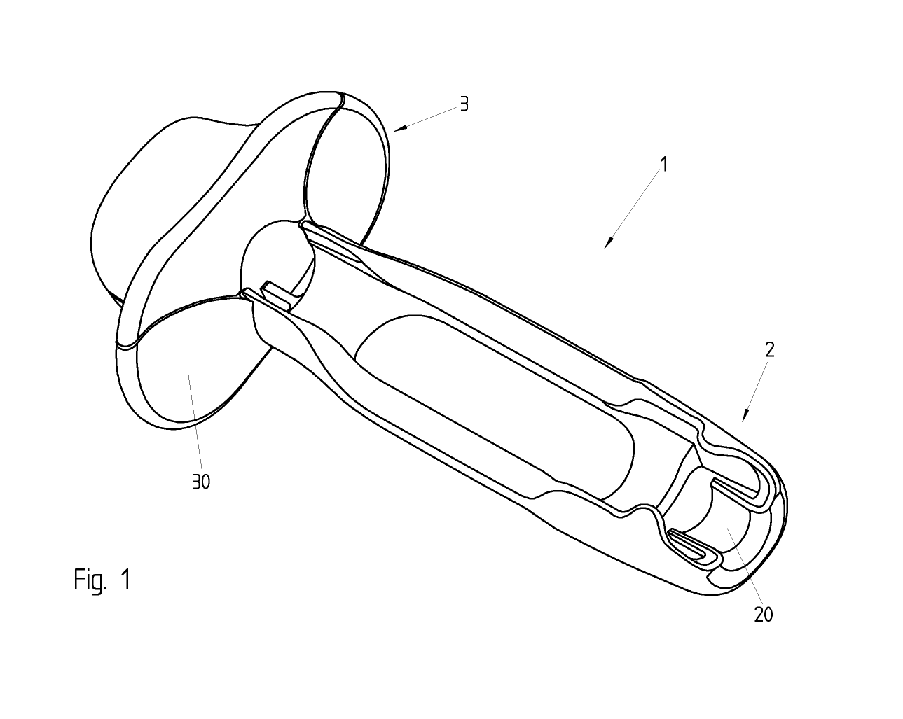

According to one embodiment illustrated in FIG. 1 by way of illustrating but non-limiting example, the accessory 1 for a syringe comprises a body capable of receiving at least partly a syringe. The body of the accessory 1 is for example elongated and comprises a distal end 2 for receiving the end of the syringe on which the syringe is located and a proximal end 3 for receiving the base of the syringe's reservoir, which comprises for example flanges or holding zones that are generally provided to enable a user or an assisted injection system to exert a bearing pressure in reaction to the injection force applied on the syringe's piston.

The distal end 2 of the accessory 1 comprises for example a bearing element 20 to hold the needle on the syringe during injection. The bearing element 20 is for example configured to bear at least partially on the tapered and/or cylindrical hub of the needle. The proximal end 3 comprises a zone 30 for application of a holding force in reaction to an injection force on the syringe's piston, for example flanges 30 enabling a user to get bearing to hold the syringe, through the accessory 1, whilst the force necessary for discharging the product to be injected is applied on the syringe's piston. The flanges 30 enable for example the user's index and middle finger to rest whilst the user otherwise pushes with the thumb on the syringe's piston.

Between the proximal end 3 and the distal end 2, the body of the accessory 1 is for example openwork to enable the user to control the quantity of product to be injected remaining in the syringe's reservoir. The openwork body of the accessory 1 also enables a user to access the syringe through these openings, for example to make the syringe turn around its longitudinal axis before a new injection and thus to orient the bevel, the elbow or the opening of the needle or of the cannula in the desired direction. According to the embodiment illustrated in the figures, the body of the accessory 1 is open, at least on part of its length, on half its circumference to allow a syringe to be inserted by translation or rotation in a plane parallel to the longitudinal axis of the accessory 1.

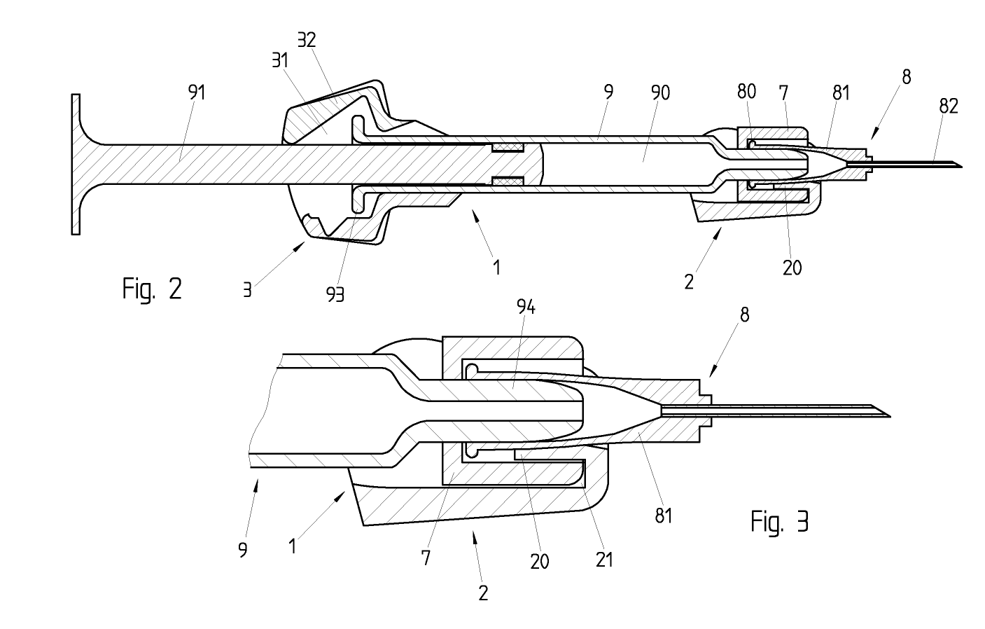

FIG. 2 is a cross section of a syringe 9 provided with an accessory 1, ready for injection or during injection. The syringe 9 comprises a reservoir 90 for containing a product to be injected and a piston 91 to discharge the product to be injected out of the reservoir 90, through the nozzle 94. A needle 8 is mounted on the nozzle 94 to allow the injection, for example hypodermically, of the discharged product.

Since the needle 8 needs to remain sterile until it is used, it is generally put into place immediately prior to the injection. To assemble it onto the syringe 9, the needle 8 is positioned on the nozzle 94 and pushed parallel to the longitudinal axis of the syringe 9 in the direction of the reservoir 90 until it locks. The needle 8 is held onto the nozzle 94 by the adhesion force between the outer wall of the nozzle 94 that is slightly conical and the inner wall of the hub 81 of the needle 8, which is also slightly conical. The tilt angle of the tapered section of the needle 8 is preferably essential equal to the tilt angle of the tapered section of the nozzle 94 with which it is in contact when the needle 8 is mounted on the syringe 9. During assembly and tightening of the needle 8 on the syringe 9, the two cones thus formed enable an adhesion to be created and the needle 8 to be held on the nozzle 94 of the syringe 9.

According to one embodiment, the needle 8 comprises for example a hub 81 of plastic material or of metal, and a tube 82, for example metallic. The conical hub 81 is of the female “luer” connector type, inspired for example from standard ISO594. The nozzle 94 of the syringe 9 having a conical geometry is of the male “luer” connector type, inspired from standard ISO594.

According to the embodiment illustrated by way of illustrating but non-limiting example, the syringe 9 is equipped with a luer lock 7 to make it easier to mount the needle 8 and give the user sufficient strength when tightening the needle 8 on the syringe 9 in order to ensure there is a good adhesion between them. The luer lock 7 is an optional cylindrical device that is for example clipped onto the nozzle 94 of the syringe 9 before the needle 8 is mounted. The luer lock 7 is for example held on the syringe 9 by a circular shoulder, not represented in the figures, formed on the circumference of the nozzle 94. The luer lock 7 further comprises on its inner wall a screw thread, also not represented in the figures, wherein for example a shoulder 80 at the bottom of the needle 8 is driven. The needle 8 is thus mounted on the syringe 9 by screwing in the luer lock 7, which has the effect of moving it with a certain force closer to the nozzle 94, until it is coupled with the syringe 9. The luer lock 7 can also be molded in a single block with the body of the syringe 9, in particular in the case of a plastic syringe.

The proximal end 3 of the accessory 1 comprises a housing 31 for receiving for example the base of the reservoir 90 and the collar 93 of the syringe 9 when the accessory 1 and the syringe 9 are assembled. The housing 31 is for example closed on the periphery of the syringe 9 and completely surrounds the base of the reservoir 90, so that the collar 93 is not accessible to a user. The housing 31 is for example formed of a cover 32 that is clipped onto the proximal end 3 of the accessory 1 after insertion of the syringe 9.

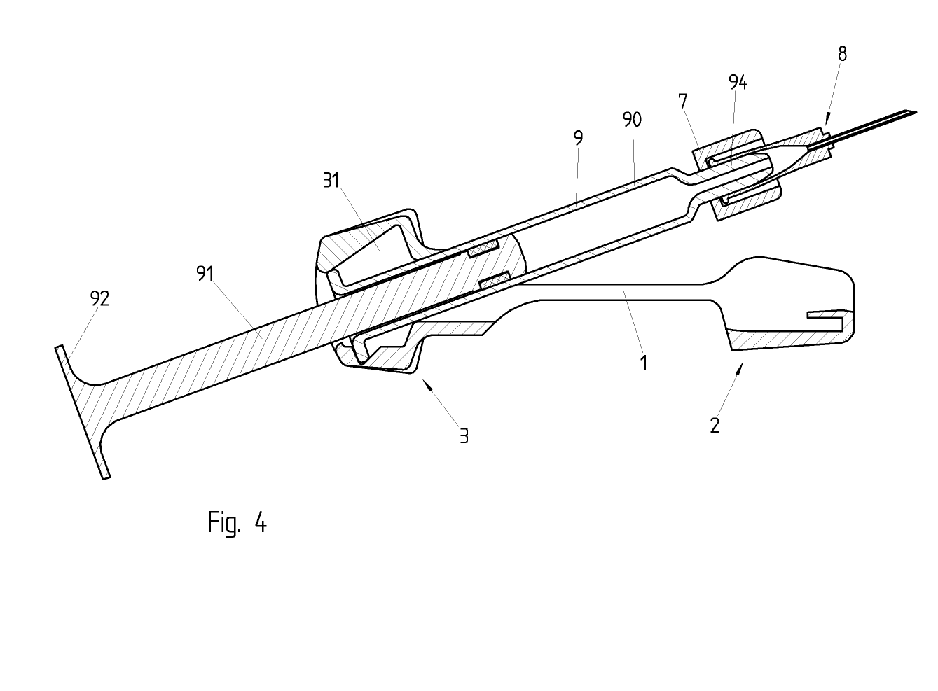

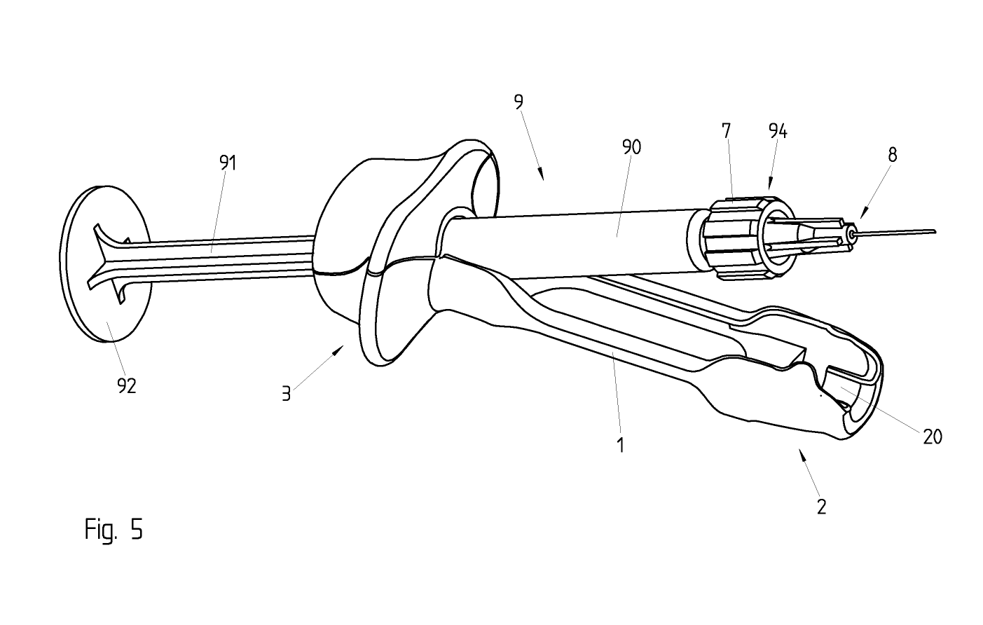

As illustrated in FIGS. 2 and 4, the housing 31 is configured for example to enable the syringe 9 to swing the syringe 9 into the accessory 1 between an initial position illustrated in FIG. 4, wherein the longitudinal axis of the syringe 9 forms an angle with the longitudinal axis of the accessory 1, and an injection position illustrated in FIG. 2, wherein the longitudinal axis of the syringe 9 is parallel to the longitudinal axis of the accessory 1. In the initial position, the nozzle 94 of the syringe is disengaged from the accessory 1 and accessible to a user, for example for mounting the needle 8. In the injection position, the needle 8 is held against the syringe 9 by the bearing element 20 of the distal end 2. The housing 31 is configured so that in the injection position, the collar 93 of the syringe 9 is not in contact with the accessory 1, in particular with the inner walls of the housing 31.

With reference to FIG. 3, which is a detailed cross section of the distal end 2 of the accessory 1 and of the nozzle 94 of the syringe 9 in the injection position, the distal end 2 comprises a bearing element 20 configured for bearing on the needle 8, for example on the hub 81 of the needle 8, when the syringe 9 and the accessory 1 are in the injection position and thus hold with a certain force the needle 8 against the syringe 9 during injection. The bearing element 20 is for example a tapered section, for example conformed to the outer wall of the hub 81 of the needle 8 to maximize the bearing surface in the injection position. The distal end 2 of the accessory 1, in particular the bearing element 20, comprises for example an opening on its periphery to enable the lateral insertion of the needle 8 in the distal end 2 of the accessory 1 when the syringe 9 is swung from the initial position into the injection position.

According to one embodiment, the opening in the distal end 2, visible for example in FIG. 1, is sized to enable the lateral insertion of a portion of the needle 8 finer than that on which the bearing element 20 bears during the injection, for example the tube or cannula and/or part of the distal end of the hub 81. The bearing element 20 is then configured for example to bear on more than 180°, for example on 200° or preferably on 240°. In the injection position, since the bearing element 20 surrounds the hub 81 of the needle 8 over more than half of its periphery, the needle 8 is locked in the distal end 2, thus preventing it from involuntarily becoming expelled from the distal end 2 during injection.

According to the embodiment illustrated by the figures, the distal end 2 comprises a housing 21 to receive a luer lock 7 when the accessory 1 and the syringe 9 are in the injection position, thus making it possible to use the accessory 1 with a syringe provided with a luer lock 7. The housing 21 is configured for example so that in the injection position, the luer lock 7 is not in contact with the accessory 1, so that no force is transmitted from the accessory 1 to the luer lock 7 during injection and that the entire holding force applied on the accessory 1 during injection is transmitted to the needle 8.

The housing 31 of the proximal end 3 is configured for example so that in the injection position, the collar 93 of the syringe 9 is not in contact with the accessory 1, in particular with the inner walls of the housing 31, so that during injection no force is transmitted directly from the accessory 1 to the reservoir 90 of the syringe, in particular to the collar 93, and that most, preferably all, of the holding force is transmitted by the accessory 1 to the needle 8 to ensure it is held onto the syringe 9.

During injection, a user exerts a pressure on the piston 91 of the syringe 9 by bearing for example with the thumb on its base 92 whilst pressing for example with the index and middle finger on the zone for application of a holding force, for example the flanges 30, of the accessory 1. The body of the accessory 1 being preferably rigid, the force exerted by the user on the flanges 30 is at least partly applied to the needle 8, for example to its hub 81, through the contact surface between the bearing element 20 of the distal end 2 and the needle 8. In the embodiments with no mechanical contact during injection between the accessory 1 on the one hand and the collars 93 and the luer lock 7 on the other hand, the force applied by the accessory 1 on the needle 8 corresponds essentially to the force applied by the user on the accessory 1.

The bearing element 20, in particular its surface that is in contact with the needle 8 in the injection position, is configured according to certain embodiments so that the force exerted by the user on the accessory 1 and which is essentially oriented parallel to the longitudinal axis of the syringe 9 results in a force applied by the accessory 1 on the needle 8 that comprises a longitudinal component oriented parallel to the longitudinal axis of the syringe 9 and a radial component oriented perpendicularly to this axis and towards the center of the syringe 9. The longitudinal component is directed in a direction opposite to the direction of the stress exerted on the needle 8 by the product discharging out of the syringe during injection, thus directly compensating at least part of this stress, whilst the radial component of the bearing force of the accessory 1 on the needle 8 reinforces the adhesion of the needle 8 on the nozzle 94 of the syringe 9.

This decomposition of the bearing force of the accessory 1 on the needle 8 into a longitudinal component and a radial component is for example achieved by the tapered element of the bearing element 20, in particular of its surface that is in contact with the needle 8 in the injection position. The tilt angle of the tapered bearing element 20 corresponds for example to the tilt angle of the outer surface of the hub 81 of the needle 8, so as to maximize the bearing surface of the accessory 1 on the needle 8.

According to other embodiments, this decomposition of the bearing force of the accessory 1 on the needle 8 into a longitudinal component and a radial component is achieved for example by the essentially cylindrical nature of the bearing element 20, in particular its surface which is in contact with the needle 8 in the injection position. This decomposition of the bearing force of the accessory 1 onto the needle 8 in a longitudinal component and a radial component is achieved for example by the lateral tightening of the hub 81 of the needle 8 by the bearing element 20, in particular its surface which is in contact with the needle 8 in the injection position.

According to an embodiment, not represented in the figures, the tapered and/or cylindrical section 20 is configured to be in contact with the shoulder 80 generally present at the base of the needle 8 when the syringe 9 and the accessory 1 are in the injection position, thus increasing the longitudinal component of the bearing force exerted by the accessory 1 on the needle 8 during the injection and thus providing a greater direct compensation of the stress exerted on the needle by the product discharged from the syringe 9.

According to the embodiment illustrated by the figures, the insertion of the luer lock 7 into the housing 21 of the distal end 2 in the injection position ensures that the accessory 1 and the syringe 9 are locked in their relative position whilst preventing the distal end 2 from moving away from the nozzle 94 since it would then be held back by the luer lock 7. The bearing element 20 thus forms with the luer lock 7 a locking mechanism of the accessory 1 in the injection position relative to the syringe 9. The locking mechanism thus prevents the accessory 1 from becoming detached from the syringe 9 during the injection. This locking mechanism is important in particular if the bearing element 20 bears for example only on half or less of the circumference of the hub of the needle 8.

FIGS. 4 and 5 show the syringe 9 equipped with the accessory 1 in an initial position, before the injection. In this position, the longitudinal axis of the accessory 1 forms an angle with the longitudinal axis of the syringe 9. The accessory 1 is then fastened to the syringe 9 by the proximal end 3 which surrounds the syringe 9 for example at the base of the reservoir 90, whilst the distal end 2 is moved away from the nozzle 94. In this initial position, the nozzle 94 of the syringe 9 is accessible, thus enabling the needle 8 to be mounted. The proximal end 3 entirely surrounds the syringe 9, so that the accessory 1 cannot inadvertently become unfastened from the syringe 9.

After the needle 8 has been mounted on the nozzle 94, the syringe 9 and/or the accessory 1 are pivoted until their longitudinal axes are aligned. The needle 8 then passes through the opening provided for this purpose in the distal end 2. The syringe 9 is then advanced in the direction of its longitudinal axis in the direction of the distal end of the accessory 1 until the bearing element 20 is in direct contact with the needle 8, for example with its hub 81, and the assembly constituted by the syringe 9 and accessory 1 finds itself in the injection position such as represented in FIG. 2.

During the translation of the syringe 9 relative to the accessory 1, the bearing element 20 is inserted for example at least partially in the luer lock 7, thus ensuring the syringe 9 and the accessory 1 are locked. The translation movement can be initiated for example by the user exerting a pressure in a similar way to the pressure exerted during the injection, i.e. by a pressure between the base 92 of the piston 91 of the syringe 9 and the zone for application of a holding force, for example the flanges 30, of the accessory 1 on the other hand.

In the examples above, the zone for application of the holding force on the accessory is constitute of flanges 30 integral to the accessory 1 and particularly adapted for example to a manual injection. According to a variant embodiment, this zone is constituted of or comprises eyes, rigid loops or any other adapted bearing device.

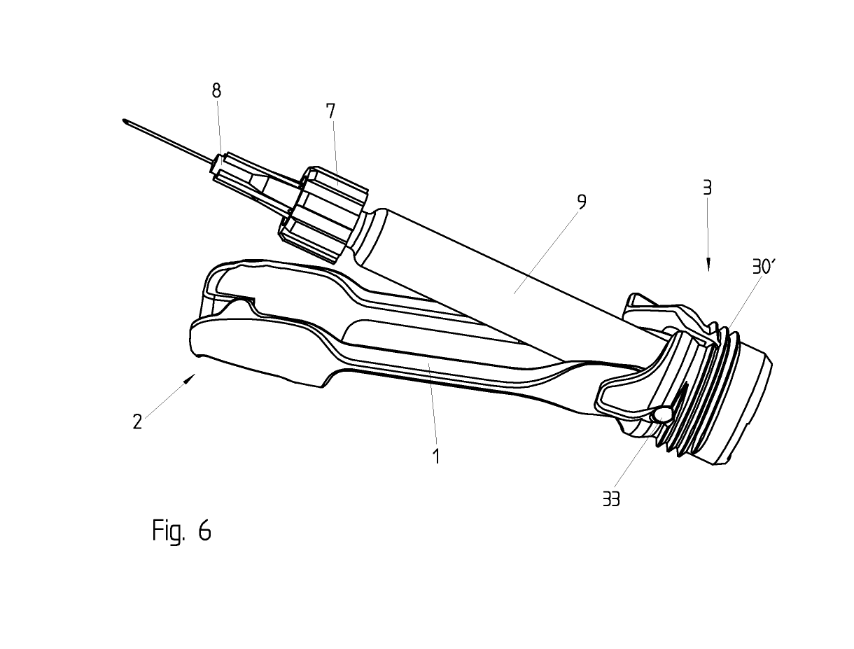

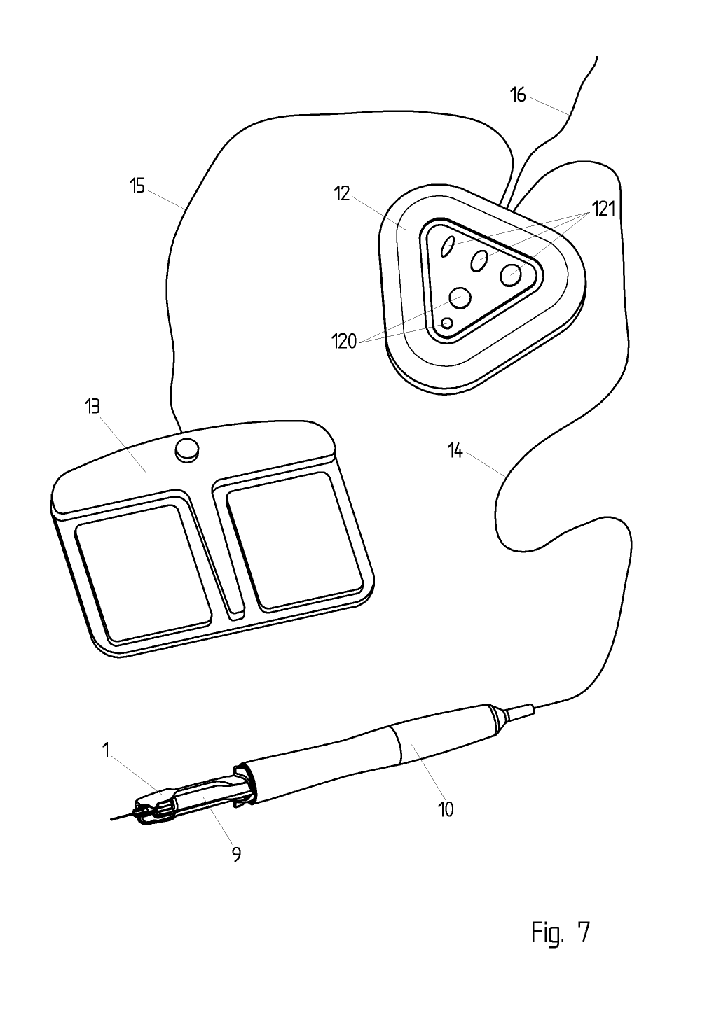

According to other embodiments illustrated in the FIGS. 6 and 7 by an illustrating but in no way limiting example, the accessory 1 for syringe is adapted for an assisted, mechanical and/or automatic, injection. According to this embodiment, the zone for application of a holding force of the accessory 1 is adapted to it being coupled to an assisted injection device, comprising for example fastening means allowing it to be fastened to an assisted injection device.

According to the example illustrated in FIG. 6, the fastening means comprise for example a screw thread 30′ in the zone for application of a holding force allowing the accessory 1 to be screwed at the extremity of a handpiece during the installation of the syringe 9 on an assisted injection device. During the injection, an injection mechanism of the assisted injection device bears on the piston of the syringe 9 with an injection force, thus causing the application of a corresponding holding force on the accessory 1 through the fastening means, for example through the screw thread 30′. The force applied on the zone for application of the holding force 30′ is then retransmitted for the main part to the needle 8, as explained previously in the case of a manual injection.

Other fastening means are conceivable in the frame of the invention for fastening the accessory 1 to an assisted injection device, in particular a handpiece of an assisted injection device. These fastening means comprise for example one or several bayonets, one or several clips and/or any other suitable fastening means. The fastening means preferably enable the accessory 1 to be removably coupled to the assisted injection device. The fastening means are preferably chosen and sized to resist the holding force applied on the accessory 1 during an injection.

The accessory 1 comprises for example an articulation 33 between its proximal end 3 and its distal end 2, enabling the distal end 2 of the accessory 1 to be moved away from the nozzle of the syringe 9 when the accessory 1 and the syringe 9 are assembled and for example fastened together on an assisted injection device. In this initial position illustrated by way of example in FIG. 6, the nozzle of the syringe 9 is accessible to allow for example the needle 8 to be fastened on the syringe 9. Before the injection, the distal end 2 of the accessory 1 is pivoted around the articulation 33 until the longitudinal axis of the syringe 9 is aligned with the longitudinal axis of the accessory 1 and the distal end 2 of the accessory 1 surrounds the needle 8 at least partially. The syringe is then for example advanced along its longitudinal axis until the bearing zone of the distal end 2 bears against the needle 8, in particular against its hub.

The manner in which the accessory 1 holds the needle 8 against the syringe 9 is perfectly similar to that described further above in relation with the accessory for a syringe adapted for manual injection, the differences between the two variants being essentially in the shape and nature of the bearing zone of a holding force, i.e. in the configuration of the proximal end 2 of the accessory 1 which is suited for it being fastened to an assisted injection device, and optionally to a particular shape of the syringe 9 adapted for its use with an assisted injection device, in particular of the reservoir and of the piston of such syringes.

FIG. 7 illustrates an assembly comprising a syringe 9 and an accessory 1 according to the invention, adapted for use with an assisted injection device, coupled with an assisted injection device.

According to the example illustrated, the assisted injection device comprises a handpiece 10, a controller case 12 and a pedal unit 13.

The handpiece 10 is preferably of a rigid material and of ergonomic shape to enable a good grip in the hand by a user during injections. The handpiece comprises for example a motor and a driving mechanism making it possible to press on the piston of the syringe 9 housed at least partially in the handpiece 10. Other embodiments are however possible in the frame of the invention. According to certain variant embodiments, the motor is lodged in the controller case in order to minimize the volume of the handpiece, and the driving mechanism actuates the piston of the syringe 9 through a flexible link connecting the controller case 12 to the handpiece 10, for example by means of a semi-rigid steel cable.

The control case 12 of the assisted injection device comprises for example a microprocessor, a memory and software lodged in the memory and that can be executed by the microprocessor, that enable it to control with precision the quantity and the rate of the product to be injected that is discharged from the syringe 9 by the precise control of the motor's movements, for example its direction of rotation, its speed of rotation and/or the number of turns executed. The controller case 12 allows for example the intensity and/or voltage of the electric current supplied to the rotating electric motor to be adjusted in order to ensure the motor has controlled movements that are regular and accurate, regardless of the encountered resistance to the injection.

The controller case 12 preferably comprises control buttons 120 allowing a user to choose a delivery rate and/or quantity of product to be injected and/or to choose between a continuous or drop-by-drop injection. The case preferably also comprises light indicators 121 and/or a digital display and/or a sound signaling device enabling a user to visually and/or audibly control the adjustments performed and/or the dosage in progress. During the injection, the software implemented in the controller case 12 ensures the motor operates regularly and accurately depending on the delivery rate and/or the quantity entered previously by the user.

The actuation of the motor is controlled for example by means of a pedal unit 13, thus enabling the user to have both hands free to guide the insertion of the syringe needle under the patient's skin and possibly the movements of the needle during the injection.

The handpiece 10, the controller case 12 and the pedal unit 13 are for example connected to one another by flexible links, for example communication and/or supply cables 14, 15 enabling a communication between these elements, in particular the communication of the command signals, of the control signals and/or of the transmission of the electrical and/or mechanical energy. The assisted injection device is preferably powered by an external electric energy source, not represented, to which it is connected for example by means of an electric power cable 16.

The handpiece 10 is configured to enable for example a syringe 9 to be at least partially received and the accessory 1 of the invention to be fastened. Prior to an injection, the accessory 1 is assembled to the syringe 9, for example by insertion of the proximal end of its reservoir in the proximal end 2 of the accessory 1, and fastened to the handpiece 10 by means of fastening means of the accessory 1, for example a screw thread not visible in FIG. 7, working with corresponding fastening means of the handpiece 10, for example an inner threading, also not visible in FIG. 7.

According to a variant embodiment, the accessory 1 alone is first fastened to the assisted injection device, then the syringe 9 is inserted in the accessory 1 and fastened by the positioning of the syringe 9 and of the accessory 1 in the injection position.

As previously explained, the accessory 1 for a syringe makes it possible to prevent any detaching of the needle, even under injection conditions usually considered difficult, for example when using fine needles, for example needles of 25G caliber or finer, and/or for the injection of products with a high viscosity, for example products having viscosities greater than or equal to 10'000 mPa·s, for example greater than or equal to 100'000 mPa·s. By using the accessory 1 of the invention, it is thus possible to inject, manually or by means of an assisted injection device, products with a high viscosity through fine needles, which makes it possible to minimize pain for the patient as an injection with fine needles is generally less painful than an injection with needles of a wider diameter. Furthermore, since the injection can take place at the usual injection speeds without risk of the needle becoming detached, this enables the user to perform the injection, manual or assisted, without risk, at regular speed, thus avoiding any pain to the patient that could result from variations in the delivery rate of the injected product. The usual injection speeds are for example comprised between 0.2 ml/min and 4 ml/min, for example between 0.5 ml/min and 2 ml/min.

1. Accessory for a syringe comprising:
a body including
a proximal end having at least one zone for application of a holding force in reaction to an injection force applied to the piston of the syringe, when the accessory is mounted onto the syringe, to discharge a product outside of said syringe; and
a distal end comprising a bearing element for bearing against a needle mounted on the nozzle of the syringe, when the accessory is mounted onto the syringe, the bearing element being arranged for bearing against the needle with a force comprising a radial component perpendicular to the longitudinal axis of the syringe and directed towards the center of said syringe,
wherein the bearing element comprises a tapered element matching a hub of the needle and is arranged to bear on the hub at the level of the junction between said hub and the nozzle of the syringe.

2. Accessory according to claim 1, wherein said proximal end comprises a housing surrounding at least part of the syringe over its entire periphery.

3. Accessory according to claim 1, wherein said proximal end comprises fastening means for fastening said accessory to a handpiece of an assisted injection device.

4.-5. (canceled)

6. Accessory according to claim 1, wherein the bearing element makes it possible to bear on the needle over at least half of the circumference of the hub of said needle.

7. Accessory according to claim 1, wherein the bearing element makes it possible to bear on the needle over at least two thirds of the circumference of the hub of said needle.

8. Accessory according to claim 1, wherein said distal end comprises an opening on its circumference allowing the needle to be inserted into said distal end in a direction perpendicular to the longitudinal axis of the syringe.

9. (canceled)

10. Assembly comprising:
a syringe comprising a reservoir for containing a product to be injected, and a piston for discharging the product out of the reservoir;
an accessory for a syringe comprising a body with a proximal end, which comprises at least one zone for application of a holding force in reaction to an injection force applied on the piston of the syringe for discharging a product from said syringe, wherein the body of the accessory further comprises a distal end comprising a bearing element arranged for bearing against a needle mounted on the nozzle of the syringe, the bearing element being arranged for bearing against said needle with a force comprising a radial component perpendicular to the longitudinal axis of the syringe and directed towards the center of said syringe,
wherein said bearing element comprises a tapered element matching a hub of the needle and that is arranged to bear on the hub at the level of the junction between said hub and the nozzle of the syringe.

11. Assembly according to claim 10, wherein the accessory is fastened to the syringe by said proximal end.

12. Assembly according to claim 10, further comprising an assisted injection device, wherein said assisted injection device comprises a handpiece for receiving the syringe and the accessory.

13. Assembly according to claim 10, wherein said zone for application of a holding force is configured for its mechanical coupling to said handpiece.

14. Method for preparing a syringe with the aim of an injection, comprising:
at least partial insertion of a syringe into an accessory according to claim 1;
alignment of the longitudinal axis of the syringe and of the longitudinal axis of the accessory; and
translation of the syringe relative to the accessory in order to lock said accessory and said syringe in an injection position.

15. Method according to claim 14, further comprising fastening said accessory to a handpiece of an assisted injection device.

16. Method according to claim 14, further comprising applying a holding force to said zone for application of a holding force in reaction to an injection force applied on the piston of the syringe for discharging a product from said syringe.

17. Method for preparing a syringe for an injection, said method comprising:
the coupling of a fine needle on a syringe and
the locking of the needle on the syringe by way of an accessory according to claim 1 in order to minimize the pain of a patient during the injection at a usual speed of a product having a high viscosity by way of said syringe.

18. Method according to claim 17, further comprising fastening said syringe and said accessory on an assisted injection device.

19. Accessory according to claim 1, wherein the distal end of the body comprises a housing for receiving at least part of a luer lock placed on the syringe without said luer lock being in contact with said distal end when the bearing element bears against the needle.
